(19) United States
(12) Patent Application Publication
(10) Pub. No.: US 2026/0335264 A1
(43) Pub. Date: Oct. 29, 2026

(54) METHOD AND COMPOSITION FOR AMELIORATING SITE PAIN DURING SUBCUTANEOUS ADMINISTRATION OF TREPROSTINIL

(71) Applicant: Ken Phares, Hillsborough, NC (US)

(72) Inventor: Ken Phares, Hillsborough, NC (US)

(21) Appl. No.: 19/638,232

(22) Filed: Apr. 3, 2026

Related U.S. Application Data
(60) Provisional application No. 63/783,707, filed on Apr. 4, 2025.

(51) Int. Cl. A61K 31/192; A61K 47/18

(52) U.S. Cl. CPC A61K 31/192; A61K 47/183

(57) ABSTRACT

A pharmaceutical composition includes a therapeutically effective concentration of treprostinil, at least an equimolar concentration of a cationic compound that forms an ionic interaction with treprostinil at or near neutral pH, and a pharmaceutically acceptable solvent. The cationic compound may be an amino acid such as a D-amino acid, L-arginine, lysine and/or histidine. In another aspect, a method of treating pulmonary arterial hypertension involves administering the pharmaceutical composition to a subject in need thereof, such as by subcutaneous administration.

### CROSS REFERENCE TO RELATED APPLICATIONS

This application claims the benefit of priority to U.S. Provisional Application No. 63/783,707, filed Apr. 4, 2025, which is hereby incorporated by reference in its entirety.

### BACKGROUND

Pulmonary arterial hypertension (PAH) is a progressive and life-threatening disease characterized by increased pressure in the pulmonary vasculature that can lead to heart failure. PAH has been previously classified as primary (idiopathic) or secondary. Currently there are a number of FDA-approved products for treating certain types of PAH. These products include treprostinil as the active ingredient, such as Remodulin® injection. There are eight dosages of Remodulin currently on the market, namely 0.1-, 0.2-, 0.4, 1-, 2.5, 5-, 10-, and 20-mg/mL of treprostinil in the acid molecular form. The formulations also include a citric acid/sodium citrate buffer, meta-cresol preservative, and sodium chloride to adjust tonicity.

A major side effect associated with subcutaneous delivery of treprostinil is site pain. In some cases, site pain is so severe that patients discontinue treatment. Some attempts to reduce site pain have included topical gels, ice packs, nonsteroidal anti-inflammatory agents, opiates, and altering the chemical structure of treprostinil with prodrugs. See generally, E. O. Jackson et al., “Pediatric subcutaneous treprostinil site maintenance and pain control strategies from the Pediatric Pulmonary Hypertension Network,” Pulmonary Circulation, Vol. 11, No. 1 pp. 1-7 (2021). Other approaches involved altering the formulation by removing the meta-cresol preservative, removing or changing the citric acid/sodium citrate buffer, or adjusting pH. However, none of these approaches has proven effective to consistently reduce site pain associated with subcutaneous delivery of treprostinil. Accordingly, there remains a need to develop techniques to ameliorate site pain associated with subcutaneous administration of treprostinil, including and especially during the treatment of PAH.

### SUMMARY OF THE INVENTION

In one aspect, a pharmaceutical composition comprises a therapeutically effective concentration of treprostinil, at least an equimolar concentration of a cationic compound that forms an ionic interaction with treprostinil at or near neutral pH, and a pharmaceutically acceptable solvent therefor. In some examples, the cationic compound is an amino acid such as L-arginine, lysine and/or histidine.

In some examples, the pharmaceutical composition has a pH ranging generally from about 5 to about 10, typically from about 5 to about 9, often from about 6 to about 8. In the case of a composition having a pH deviating from physiological pH (e.g., from about 5 to about 6 or from about 8 to about 10), a non-buffered composition may, in some embodiments, reduce localized and/or transient pH gradients upon administration.

In some examples, the concentration of the cationic compound is at least twice that of treprostinil.

In some examples, the cationic compound has a net positive charge at pH≥5.

In another aspect, a method of treating pulmonary arterial hypertension comprises administering to a subject in need thereof a pharmaceutical composition comprising a therapeutically effective concentration of treprostinil, at least an equimolar concentration of a cationic compound that forms an ionic interaction with treprostinil at or near neutral pH, and a pharmaceutically acceptable solvent therefor. In some examples, the pharmaceutical composition is administered subcutaneously.

These and other aspects and features are described more fully in the following detailed description.

### DETAILED DESCRIPTION

The terminology used herein is for the purpose of describing particular embodiments only and is not intended to be limiting of or to the disclosure. As used herein, the singular forms “a”, “an” and “the” are intended to include the plural forms as well, unless the context clearly indicates otherwise.

The term “about” when used before a numerical designation, e.g., temperature, time, amount, concentration, and such other, including a range, indicates approximations that may vary by (+) or (−) 10%, 5%, 1%, or any subrange or a subvalue therebetween. In one example, the term “about” when used with regard to a dose amount means that the dose may vary by +/−10%.

“Comprising” or “comprises” is intended to mean that the compositions and methods include the recited elements, but not excluding others.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure.

“Consisting of” shall mean excluding more than trace elements of other ingredients and substantial method steps. Embodiments defined by each of these transition terms are within the scope of this disclosure.

The expressions “wt. %” and “wt.-ppm” are used herein to designate weight percentages and weight and parts per million, respectively. Unless otherwise clear from context, all percentages described herein are expressed as weight percent based on the total weight of a composition.

The term “substantially,” as used herein, refers to an extent of at least 95%. For example, the phrase “substantially all” may be replaced by “at least 95%.”

The terms “optional” or “optionally,” as used herein, mean that the subsequently described event or circumstance may or may not occur and that the description includes instances where the event or circumstance occurs and instances in which it does not.

The term “salt,” as used herein, refers any positive counterion to a negative charge on a disclosed compound and any negative counterion to a positive charge on the disclosed compound.

The term “ionic interaction,” as used herein, is synonymous with ionic bonding and refers to chemical bonding involving electrostatic attraction between oppositely charged ions, or between two atoms with sharply different electronegativities. Ions are atoms (or groups of atoms) with an electrostatic charge. Atoms that gain electrons make negatively charged ions referred to as anions. Atoms that lose electrons make positively charged ions referred to as cations.

The terms “treating” or “treatment,” as used herein, refer to relieving, reducing or alleviating at least one symptom in a subject or effecting a delay of progression of a disease. For example, treatment can be the diminishment of one or several symptoms of a disorder or complete eradication of a disorder. The terms “treating” and “treatment” are inclusive of the prevention of at least one symptom associated with or caused by a disease or disorder.

### Treprostinil

Treprostinil is a tricyclic benzidine analog of epoprostenol with several advantages such as a longer half-life (about 4 h) and chemical stability at room temperature in solution. The chemical structure of treprostinil is shown below:

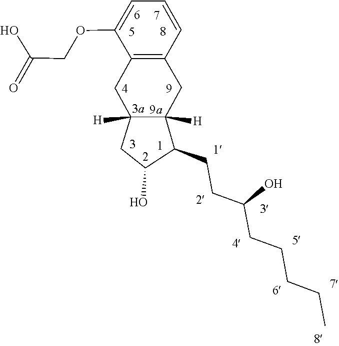 [SMILES: CCCCC[C@H](O)CC[C@@H]1[C@H]2Cc3cccc(OCC(=O)O)c3C[C@H]2C[C@H]1O]

The pKa of treprostinil is about 3.8, resulting in the compound having a negative charge (owing to its carboxylate functional group) when formulated at or near neutral pH. It is postulated that site pain results from ionic interaction between treprostinil and the prostacyclin (IP) receptor in the subcutaneous tissue. See A. L. Tsai et al., Eicosanoids, 2 (1989) 131. The structure of prostacyclin is depicted below:

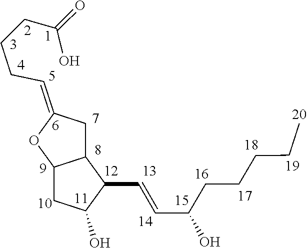 [SMILES: CCCCC[C@H](O)/C=C/[C@@H]1C2C/C(=C/CCCC(=O)O)OC2C[C@H]1O]

While not wanting to be bound by theory, it is believed that by providing a sufficient (at least equimolar vis-à-vis treprostinil) concentration of a cationic compound in a pharmaceutical composition, treprostinil may bind to the cationic compound through ionic interaction (ionic bonding). This ionic interaction in turn may prevent treprostinil from binding to the prostacyclin (IP) receptor in the subcutaneous tissue, thereby avoiding or ameliorating site pain. Once the bound treprostinil is absorbed into the blood stream, the higher ionic strength of the blood may suppress ionic interaction between treprostinil and the cationic compound, resulting in ionic dissociation that allows the free treprostinil to interact with the IP receptor.

### Cationic Compound

Various cationic compounds may be used in the pharmaceutical compositions described herein, including but not limited to amino acids (D/L) that have a net positive charge at neutral pH, such as L-arginine, lysine and histidine, as well as various other small molecules that are pharmaceutically acceptable for parenteral administration and possess a net positive charge at neutral pH. Non-limiting examples of D-amino acids include D-aspartic acid (D-Asp) and D-serine (D-Ser). In some examples, L-arginine may be used as the cationic compound. L-arginine has an excellent safety profile and has been approved by the FDA for intravenous administration up to doses of 30 grams. One of the three pKa's of L-arginine is the guanidinium side chain, which is 12.5. Therefore, at or near neutral pH, L-arginine has a net positive charge, rendering it particularly well suited to bind to the net negative carboxylate of treprostinil.

The concentration of the cationic compound in the formulation should be at least equimolar vis-à-vis treprostinil to obtain the intended ionic interactions as described herein. In some examples, the cationic compound may be present in a molar excess to minimize occurrences of unbound carboxylate groups of the treprostinil molecules. There is no particular upper limit on the concentration of the cationic compound provided that it does not interfere with the delivery of treprostinil or properties of the pharmaceutical composition. The concentration of cationic compound used may be, for example, 2, 3, 4, 5, 6, 7, 8, 9, or 10 times that of treprostinil in the pharmaceutical composition.

### Pharmaceutical Compositions

Pharmaceutical compositions may include treprostinil or a pharmaceutically acceptable salt, solvate, hydrate, clathrate or polymorph thereof, and one or more pharmaceutically acceptable excipients and/or solvents/carriers. The pharmaceutical compositions optionally contain one or more additional therapeutic agents.

Pharmaceutically acceptable excipients and carriers include pharmaceutically acceptable substances, materials and vehicles. Non-limiting examples of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, sweetening agents, flavoring agents, coloring agents, stabilizers, preservatives, antioxidants, antimicrobial agents, antibacterial agents, antifungal agents, adjuvants, encapsulating materials and coating materials. Non-limiting examples of preservatives that may be used include m-cresol, benzyl alcohol, chlorobutanol, methyl-paraben, propyl-paraben, phenol, phenoxyethanol, benzalkonium chloride, benzethonium chloride and thimerosal. The use of such excipients in pharmaceutical formulations is known in the art. For example, conventional vehicles and carriers include without limitation oils (e.g., vegetable oils, such as sesame oil), aqueous solvents (e.g., saline and phosphate-buffered saline), and solvents (e.g., dimethyl sulfoxide and alcohols such as ethanol and glycerol). Except insofar as any conventional excipient or carrier is incompatible with the active ingredient (for purposes of the content of a pharmaceutical composition, the term “active ingredient” encompasses a prodrug), the disclosure encompasses the use of conventional excipients and carriers in treprostinil formulations. See, e.g., Remington: The Science and Practice of Pharmacy, 21st Ed., Lippincott Williams & Wilkins, Philadelphia, Pennsylvania (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press LLC (2004).

The compositions may be presented, for example, in unit dosage form as a single dose in which all active and inactive ingredients are combined in a suitable system, and components need not be mixed to form the composition to be administered. The unit dosage form may contain an effective dose, or an appropriate fraction thereof, of treprostinil. In some examples, treprostinil and the cationic compound may be formulated in a pharmaceutically acceptable solvent, such as sterile water, along with any optional excipients to prepare a composition suitable for subcutaneous administration.

### Treatment Methods

Treprostinil has a variety of prostacyclin-like effects. Treprostinil may promote vasodilation, inhibit platelet activation and aggregation, inhibit thrombus formation, stimulate thrombolysis, inhibit atherogenesis, inhibit cell proliferation, inhibit angiogenesis, promote endothelial cell membrane remodeling, reduce inflammation, and provide cytoprotection. The pharmaceutical formulations described herein may be used to treat a wide variety of conditions, including without limitation: pulmonary hypertension, portopulmonary hypertension, pulmonary fibrosis, interstitial lung disease, ischemic diseases (e.g., myocardial ischemia, ischemic stroke, peripheral vascular disease (including peripheral arterial disease), ischemia of a limb, Raynaud's phenomenon (including Raynaud's disease and Raynaud's syndrome), scleroderma (including systemic sclerosis) and renal insufficiency), ischemic ulcers (e.g., digital ulcers), cardiovascular disease (e.g., coronary artery disease), heart failure (e.g., congestive heart failure), conditions requiring anticoagulation (e.g., post myocardial infarction and post cardiac surgery), atherogenesis (e.g., atherosclerosis), thrombotic microangiopathy, vein occlusion (e.g., central retinal vein occlusion), hypertension (e.g., preeclampsia), diabetic vasculopathy, extracorporeal circulation, inflammatory diseases (e.g., chronic obstructive pulmonary disease and psoriasis), reproduction and parturition, conditions of unregulated cell growth (e.g., tumors and cancers), cell/tissue preservation, and other therapeutic areas where prostacyclin or treprostinil treatment may provide benefit.

Treprostinil may be used in conjunction with an additional therapeutic agent(s) to treat any condition responsive to treatment with prostacyclin or treprostinil. As a non-limiting example, to treat a vascular (e.g., cardiovascular) disorder treprostinil may be used in combination with a vascular (e.g., cardiovascular) therapeutic, such as an antiplatelet agent, a phosphodiesterase inhibitor, a calcium channel blocker or an endothelial antagonist, or any combination thereof.

In some examples, the treprostinil formulations described herein may be used to treat pulmonary hypertension. An additional therapeutic agent (e.g., a vasoactive agent, a diuretic and/or an anticoagulant) optionally may be administered to treat pulmonary hypertension.

Pulmonary hypertension is an increase of blood pressure in the lung vasculature, including the pulmonary artery, pulmonary vein and pulmonary capillaries. Thus, pulmonary hypertension encompasses pulmonary arterial hypertension (PAH) and pulmonary venous hypertension (PVH) (e.g., congestive heart failure). More broadly, pulmonary hypertension encompasses: WHO Group I—pulmonary arterial hypertension, including idiopathic PAH, heritable PAH (e.g., BMPR2, ALK1 and endoglin [with or without hereditary hemorrhagic telangiectasia]), drug- and toxin-induced PAH, PAH associated with various conditions (e.g., connective tissue disease, HIV infection, portal hypertension, congenital heart disease, schistosomiasis, and chronic hemolytic anemia [e.g., sickle cell disease]), persistent pulmonary hypertension of the newborn, pulmonary veno-occlusive disease (PVOD), and pulmonary capillary hemangiomatosis (PCH); WHO Group II-pulmonary hypertension owing to left heart disease, including systolic dysfunction, diastolic dysfunction and valvular heart disease; WHO Group III-pulmonary hypertension owing to lung disease and/or hypoxia, including chronic obstructive pulmonary disease (COPD), interstitial lung disease, other pulmonary diseases with mixed restrictive and obstructive pattern, sleep-disordered breathing, alveolar hypoventilation disorders, chronic exposure to high altitude, and developmental abnormalities; WHO Group IV—chronic thromboembolic pulmonary hypertension (CTEPH); and WHO Group V—pulmonary hypertension with unclear multifactorial mechanisms, including hematologic diseases (e.g., myeloproliferative disease and splenectomy), systemic diseases (e.g., sarcoidosis, pulmonary Langerhans cell histiocytosis, lymphangioleiomyomatosis, neurofibromatosis and vasculitis), metabolic disorders (e.g., glycogen storage disease, Gaucher disease and thyroid diseases), and other causes (e.g., tumoral obstruction, fibrosing mediastinitis and chronic renal failure on dialysis).

The therapeutically effective concentration and frequency of administration to treat pulmonary hypertension may depend on various factors, including the type of pulmonary hypertension, the severity of the condition, the mode of administration, the age, body weight, general health, gender and diet of the subject, and the response of the subject to the treatment, and can be determined by the treating physician. In certain embodiments, the effective dose of treprostinil per day is about 0.1 to about 100 mg, about 0.1 to about 50 mg, about 0.5 to about 50 mg, about 0.5 to about 25 mg, about 0.5 to about 10 mg, about 1 to about 10 mg or about 1 to about 5 mg as a continuous subcutaneous infusion as deemed appropriate by the treating physician. In further embodiments, the effective dose of treprostinil per day is about 0.001 to about 2 mg/kg, about 0.005 to about 1 mg/kg, about 0.01 to about 0.5 mg/kg or about 0.01 to 0.1 mg/kg body weight as a continuous subcutaneous infusion as deemed appropriate by the treating physician.

In some embodiments, treprostinil is administered in a single dose or in multiple doses daily (including one, two, three or more times daily), every two days, every three days, weekly, every 2 weeks, every 3 weeks, monthly, every 6 weeks, every 2 months or every 3 months, or as deemed appropriate by the treating physician. In further embodiments, treprostinil is administered as a continuous subcutaneous infusion as the dosing regimen. In certain embodiments, a therapeutically effective concentration of treprostinil is administered over a period of at least 2 weeks, 3 weeks, 1 month, 1.5 months, 2 months, 3 months, 4 months, 5 months, 6 months or longer.

### Example

The following prophetic example is given to illustrate certain aspects of the present disclosure and should not be construed as limiting the scope of the invention as other equivalent embodiments will be apparent in view of the present disclosure and appended claims.

A formulation adapted for subcutaneous delivery may be prepared that includes 1 mg/mL treprostinil in sterile water, which corresponds to 2.6 mM of treprostinil. The composition may have an approximately 2-fold concentration excess of L-arginine compared to treprostinil, equivalent to 5.2 mM of L-arginine or 0.45 mg/mL. This excess concentration of L-arginine helps to ensure that there is no unbound treprostinil in the formulation that may cause site pain during subcutaneous administration. Sodium chloride or other charged tonicity agent is not present in this particular formulation because it may suppress ionization of treprostinil and L-arginine and favor dissociation of the charged compounds. Tonicity agents that are uncharged, such as sucrose or dextrose, optionally may be used. The illustrated composition does not include a buffer but one optionally may be added. The composition optionally includes a preservative such as meta-cresol. However, a preservative may not be needed because the excess L-arginine with its guanidinium positive charge may provide sufficient antimicrobial activity.

It should be understood that the features of the disclosure are susceptible to modifications and/or substitutions without departing from the spirit and scope of this disclosure. The specific embodiments illustrated and described herein are for illustrative purposes only, and not limiting of the invention as defined by the appended claims.

What is claimed is:

1. A pharmaceutical composition comprising a therapeutically effective concentration of treprostinil, at least an equimolar concentration of a cationic compound that forms an ionic interaction with treprostinil at or near neutral pH, and a pharmaceutically acceptable solvent therefor.

2. The pharmaceutical composition of claim 1, wherein the cationic compound comprises an amino acid selected from the group consisting of D-amino acids, L-arginine, lysine and histidine.

3. The pharmaceutical composition of claim 2, wherein the cationic compound comprises L-arginine.

4. The pharmaceutical composition of claim 1, wherein the pH of the composition ranges from about 5 to about 10.

5. The pharmaceutical composition of claim 4, wherein the pH ranges from about 6 to about 8.

6. The pharmaceutical composition of claim 1, wherein the concentration of the cationic compound is at least twice the concentration of treprostinil.

7. The pharmaceutical composition of claim 1, wherein the cationic compound has a net positive charge at pH≥5.

8. The composition of claim 1, wherein the concentration of treprostinil ranges from about 0.1 to about 20 mg/mL.

9. The composition of claim 1, further comprising an excipient selected from the group consisting of a buffer, a tonicity agent, a preservative and a combination thereof.

10. A method of treating pulmonary arterial hypertension comprising administering to a subject in need thereof a pharmaceutical composition comprising a therapeutically effective concentration of treprostinil, at least an equimolar concentration of a cationic compound that forms an ionic interaction with treprostinil at or near neutral pH, and a pharmaceutically acceptable solvent therefor.

11. The method of claim 10, wherein the composition is administered subcutaneously.

12. The method of claim 10, wherein the composition is administered to provide a dose of treprostinil ranging from about 0.001 to about 2 mg/kg of body weight of the subject.

13. The method of claim 10, wherein the composition is administered to provide a dose of treprostinil ranging from about 0.005 to about 1 mg/kg of body weight of the subject.

14. The method of claim 10, wherein the composition is administered to provide a dose of treprostinil ranging from about 0.01 to about 0.1 mg/kg of body weight of the subject.

15. The method of claim 10, wherein the cationic compound comprises an amino acid selected from the group consisting of L-arginine, lysine and histidine.

16. The method of claim 15, wherein the cationic compound comprises L-arginine.

17. The method of claim 10, wherein the pH of the composition ranges from about 5 to about 10.

18. The method of claim 17, wherein the pH ranges from about 6 to about 8.

19. The method of claim 10, wherein the concentration of the cationic compound is at least twice the concentration of treprostinil.

20. The method of claim 10, wherein the cationic compound has a net positive charge at pH≥5.
